SEQUENCE LISTING (3 sequences)

SEQ ID NO: 1 - DNA, 22 bp, Artificial Sequence
  Defensin B1 mRNA Forward Primer
aatgccttcaacatggaggatt

SEQ ID NO: 2 - DNA, 28 bp, Artificial Sequence
  Defensin B1 mRNA Reverse Primer
ttacaggttccctgtagtttggtattag

SEQ ID NO: 3 - DNA, 26 bp, Artificial Sequence
  Defensin B1 mRNA Probe
tgtctccgctccagctgcccatamra

(19) United States
(12) Patent Application Publication
(10) Pub. No.: US 2006/0211672 A1
(43) Pub. Date: Sep. 21, 2006

(54) Use of estrogen receptor-beta selective agonists for radiation-or chemotherapy-induced mucositis and radiation cystitis

(75) Inventors: William Jacobson, North Wales, PA (US); Heather A. Harris, Phoenixville, PA (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/353,611

(22) Filed: Feb. 14, 2006

Related U.S. Application Data
(60) Provisional application No. 60/653,376, filed on Feb. 16, 2005.

(51) Int. Cl. A61K 31/428; A61K 31/423; A61K 31/56; A61K 31/4184

(52) U.S. Cl. 514/182; 514/367; 514/375; 514/394

(57) ABSTRACT

The present invention provides a method of treating or inhibiting mucositis or radiation cystitis using an ERβ selective ligand. The present invention also provides compositions, including pharmaceutical compositions, containing the ERβ selective ligand and a traditional medicament for mucositis or radiation cystitis.

### CROSS REFERENCE TO RELATED APPLICATIONS

This application claims priority benefit of U.S. Provisional Application Ser. No. 60/653,376 filed Feb. 16, 2005, the entire disclosure of which is incorporated by reference herein.

### FIELD OF THE INVENTION

The present invention relates, in part, to methods for treating radiation- or cytotoxic agent-induced mucositis and radiation cystitis, or symptoms thereof, comprising providing to a subject an effective amount of an ERβ selective ligand. In some embodiments, the ERβ selective ligand is applied topically. In some further embodiments, the ERβ selective ligand is non-uterotrophic, non-mammotrophic, or non-uterotrophic and non-mammotrophic. The present invention further relates to kits for treating radiation- or cytotoxic agent-induced mucositis and radiation cystitis, or symptoms thereof, and the like.

### BACKGROUND OF THE INVENTION

It is common in medicine today to use x-rays and/or chemotherapeutics for diagnostic and therapeutic purposes. While this serves a beneficial medical purpose, x-rays and cytotoxic agents such as chemotherapeutics can also have harmful side effects for the patient to whom the x-rays and chemotherapeutics are directed, the medical workers who must administer them, and the workers that develop/produce such agents on a day-to-day basis. People may also be exposed to x-rays and/or cytotoxic agents without their knowledge. Accidental or unintended exposure to x-rays and/or chemotherapeutics can cause harmful side-effects. For example, industrial accidents expose workers and/or users to harmful radiation and/or cytotoxic agents. A prime example of the potential effects of such an accident is the incident in 1986 at the nuclear power plant in Chernobyl in the former Soviet Union. Exposure to massive amounts of radiation immediately killed 32 plant workers and firefighters. Thousands more died later from effects of the accident. The Ukrainian government now says hundreds of thousands of people suffer from Chernobyl-related illnesses.

Another notorious industrial accident occurred at a chemical plant in Bhopal, India where, in 1984, methylisocyanate (MIC) and other reaction products, in liquid and vapor form, escaped from the plant into the surrounding areas. It is been estimated that at least 3000 people died as a result of this accident. Since that date, at least 12,000 more people have died from complications, and 120,000 remain chronically ill.

In the aftermath of recent terrorist attacks, there has also been renewed concern about the damage that could be caused by a terrorist bomb, such as a “dirty bomb” incorporating nuclear waste material and/or cytotoxic agents. While the actual destruction caused by such a “dirty bomb” might be minor, the hazards of having radioactive and/or cytotoxic agent widely dispersed around an unprotected population center could be immense. The exposure to such radiation and/or cytotoxic agents can lead to significant medical problems.

Cytotoxic chemotherapy and radiation therapy are common treatments for many types of cancer. It is well known that these treatments have significant side effects, including mucositis. About 15% to 40% of patients receiving standard-dose chemotherapy may experience some mucositis, while more than 70% of patients receiving higher doses of chemotherapy in combination with radiation, or radiation directed at the head and neck, will experience mucositis. (Sonis S T. Oral complications. Cancer Med. 2000; 5:2371-79). Certain chemotherapy drugs have been linked to mucositis and include 5-Fluorouracil, 6-Mercaptopurine, 6-Thioguanine, Actinomycin D, Amsacrine, Bleomycin sulfate, Cytarabine, Daunomycin, Docetaxel, Doxorubicin, Etoposide, Floxuridine, Hydroxyurea, Idarubicin, Methotrexate, Mithramycin, Mitomycin C, Mitoxantrone, Paclitaxel, Procarbazine hydrochloride, Vinblastine sulfate, Vincristine sulfate, and Vinorelbine. (See Dorr R T, VonHoff D D. Cancer chemotherapy Handbook, 2nd ed. Norwalk, Conn.: Appleton and Lange; 1994).

Mucositis is the swelling, irritation, and ulceration of the cells that line the digestive tract. Stomatitis is a form of mucositis that occurs in the stomach. Once thought to be simple and direct consequence of epithelial damage and loss of barrier function, development of mucositis is now appreciated to be a complex process that involves multiple cell types and signaling pathways. The pathobiology of mucositis is reviewed in Sonis S T [Nature Reviews Cancer. 2004; 4(4):277-284]. These cells reproduce rapidly and have a shorter life span than other cells in the body. Because neither chemotherapy agents nor radiation differentiates between healthy cells and cancer cells, they can quickly destroy digestive tract cells, breaking down the protective lining and leaving them inflamed, irritated, and swollen. Mucositis can develop in a variety of epithelial tissues, such as the alimentary canal (oral cavity, esophagus, stomach, small/large intestine, rectum), and can be aggravated by nausea and vomiting. Symptoms of mucositis include redness, dryness, or swelling of the mouth, burning or discomfort when eating or drinking, open sores in the mouth and throat, abdominal cramps, or tenderness, rectal redness or ulcers. The complications of mucositis can be severe enough to limit the dose of radiation or chemotherapy administered, thus possibly compromising efficacy of the cancer therapy. In addition, mucositis symptoms cause significant morbidity often leading to obligatory opioid analgesic use and the requirement for parenteral nutrition. For a review of this condition, see the following articles: Sonis S T, et al Cancer 2004; 100(9 Suppl):1995-2025; Rubenstein E B, et al, Cancer 2004; 100(9 Suppl):2026-46. Radiation exposure to the pelvic area can also lead to the development of radiation cystitis, a serious bladder condition with long-term consequences.

A number of agents inhibit the development of mucositis in preclinical animal models. These include epidermal growth factor [McKenna K J, et al, Surgery 1994; 115(5):626-32.], IL-11 [Gibson R J, et al, Digestive Diseases & Sciences 2002; 47(12):2751-7; Sonis S T, et al, Oral Oncology 2000; 36(4):373-81], keritinocyte growth factor [Farrell C L, et al, Cancer Research 1998; 58(5):933-9.], short chain fatty acids [Ramos M G, et al, Nutrition & Cancer 1997; 28(2):212-7.]. However, recent clinical practice guidelines [Rubenstein E B, et al Cancer 2004; 100(9 Suppl):2026-46] suggest there is a paucity of preventative and treatment therapies for oral and gastrointestinal mucositis, and thus there is a significant unmet medical need for new therapies.

Current treatments for mucositis or the treatments of mucositis include acyclovir, allopurinol mouthrinse, amifostine, antibiotic pastille or paste, benzydamine, camomile, chlorhexidine, clarithromycin, folinic acid, glutamine, GM-CSF, hydrolytic enzymes, ice chips, oral care, pentoxifyline, povidone, prednisone, propantheline, prostaglandin, sucralfate and traumeel. The effectiveness of these treatments varies greatly.

Cystitis is an irritation of the bladder not caused by a urinary tract infection. Radiation cystitis may result from radiation therapy for primary neoplasms or other malignancies. In some patients, however, a severe cystitis occurs in either an acute or delayed form. In acute radiation cystitis, oedema, hyperaemia, petechiae, and ulceration of the bladder wall develop. Clinically, symptoms of bladder infection such as frequency and dysuria as well as haematuria become manifest. Delayed radiation cystitis develops even up to 4 years following radiation exposure, depending on the dose and host susceptibility. Causes of radiation cystitis include radiation therapy to the pelvis area, chemotherapy with certain types of medications, and other irritants. Symptoms are similar to those caused by a urinary tract infection.

To date, current treatments for radiation cystitis include simple bladder irrigation, cystodiathermy, oral, parenteral and intravesical agent, hyperbaric oxygen therapy, hydrodistension, internal iliac embolisation, urinary diversion and cystectomy.

Estrogens have been shown to have anti-inflammatory properties in a number of preclinical models [Vegeto E, et al, Proceedings of the National Academy of Sciences of the United States of America 2003; 100(16):9614-9619; Harnish D C, et al, American Journal of Physiology Gastrointestinal & Liver Physiology 2004; 286(1):G118-G125.]. Estrogens can inhibit NFκB activity, a transcription factor central to the inflammation cascade [Tzagarakis-Foster C, et al, Journal of Biological Chemistry 2002; 277(47):44772-44777; Evans M J, et al, Circulation Research 2001; 89(9):823-830], and which may play a role in mucositis.

Estrogens exert their actions in cells by binding to receptors, two of which are known. The second form of the estrogen receptor (ER) was recently discovered [Kuiper, et al. (1996) Proceedings of the National Academy of Sciences of the United States of America 93, 5925-5930] and this protein has been designated ERβ to distinguish it from the previously known form, now called ERα. Early studies on the tissue distribution of ERβ suggested it was a good drug target and there was much initial optimism about its clinical utility [Nilsson S, et al, Trends in Endocrinology & Metabolism 1998; 9(10):387-395.]. Understanding the relative contributions of ERα and ERβ to estrogen physiology has recently been advanced by the in vivo profiling of ERα and ERβ selective agonists [Harris H A, et al, Endocrinology 2002; 143(11):4172-4177; Harris H A, et al, Endocrinology 2003; 144(10):4241-9.]. These studies clearly show that ERα mediates the effects of estrogens on the uterus, skeleton and vasomotor instability. ERβ selective agonists, however, are active in several preclinical models of inflammation and have a dramatic positive effect on the colonic epithelium. Additionally, it has recently been shown that ERβ is the predominant receptor form in the oral mucosa. [Valimaa H, et al, J Endocrinol. 2004; 180(1):55-62].

Accordingly, there is a need to provide protection against medical conditions caused or exacerbated by exposure to radiation or cytotoxic agents, be it as a part of a planned medical regimen, accidental or unintended exposure to radiation or cytotoxic agents, or malicious events such a terrorist attack. The methods described herein help meet current needs for new and more effective treatments for treating mucositis and radiation cystitis induced by radiation- or cytotoxic agents.

### SUMMARY OF THE INVENTION

In some embodiments, the present invention provides methods of treating or inhibiting mucositis in a subject in need thereof, said mucositis induced by exposure to a cytotoxic agent or to radiation, the method comprising providing to said subject an effective amount of an ERβ selective ligand. In some embodiments, the mucositis is oral mucositis, gastrointestinal mucositis, or rectal mucositis.

In some further embodiments, the present invention provides methods of treating or inhibiting radiation cystitis in a subject, said radiation cystitis induced by exposure to a cytotoxic agent or to radiation, the method comprising providing to said subject an effective amount of an ERβ selective ligand.

In some further embodiments, the present invention provides methods of treating at least one symptom of exposure of a subject to a cytotoxic agent or to radiation, the method comprising providing to said subject an effective amount of an ERβ selective ligand. In some embodiments, the symptom is selected from the group consisting of dysuria, haematuria, oedema, hyperaemia, petechiae, and ulceration of the bladder. In some further embodiments, the symptom is selected from the group consisting of redness, dryness, or swelling of the mouth, burning or discomfort when eating or drinking, open sores in the mouth and throat, abdominal cramps, rectal redness or ulcers.

In some further embodiments, the present invention provides methods of treating or inhibiting radiation cystitis in a subject suspected of being exposed to a cytotoxic agent or to radiation, the method comprising providing to said subject an effective amount of an ERβ selective ligand.

In some further embodiments, the present invention provides methods of treating or inhibiting mucositis in a subject suspected of being exposed to a cytotoxic agent or to radiation, the method comprising providing to said subject an effective amount of an ERβ selective ligand.

In some further embodiments, the present invention provides methods of treating or inhibiting mucositis in a subject in need thereof, said mucositis induced by exposure to a cytotoxic agent or to radiation, the method comprising administering to said subject escalating doses of an ERβ selective ligand.

In some of each of the foregoing embodiments, the ERβ selective ligand is applied topically. In some of each of the foregoing embodiments, the ERβ selective ligand is non-uterotrophic, non-mammotrophic, or non-uterotrophic and non-mammotrophic.

In some embodiments of the foregoing methods, the subject is a human. In some further embodiments of the foregoing methods, the exposure to a cytotoxic agent or to radiation is attendant to a therapeutic or diagnostic procedure. In some further embodiments of the foregoing methods, the exposure to a cytotoxic agent or to radiation is accidental. In some further embodiments of the foregoing methods, the exposure to a cytotoxic agent or to radiation is as a result of an industrial accident or a terrorist incident.

In some further embodiments of the foregoing methods, methods further comprise the administration of an effective amount of at least one traditional medicament. In some such embodiments, the traditional treatment is administered to the subject contemporaneously with the non-uterotropic, non-mammotrophic ERβ selective ligand.

### DESCRIPTION OF THE INVENTION

In some embodiments, the present invention provides methods of treating or inhibiting mucositis in a subject in need thereof, said mucositis induced by exposure to a cytotoxic agent or to radiation, the method comprising providing to said subject an effective amount of an ERβ selective ligand. In some embodiments, the mucositis is oral mucositis, gastrointestinal mucositis, or rectal mucositis.

In some further embodiments, the present invention provides methods of treating or inhibiting radiation cystitis in a subject, said radiation cystitis induced by exposure to a cytotoxic agent or to radiation, the method comprising providing to said subject an effective amount of an ERβ selective ligand.

In some further embodiments, the present invention provides methods of treating at least one symptom of exposure of a subject to a cytotoxic agent or to radiation, the method comprising providing to said subject an effective amount of an ERβ selective ligand. In some embodiments, the symptom is selected from the group consisting of dysuria, haematuria, oedema, hyperaemia, petechiae, and ulceration of the bladder. In some further embodiments, the symptom is selected from the group consisting of redness, dryness, or swelling of the mouth, burning or discomfort when eating or drinking, open sores in the mouth and throat, abdominal cramps, rectal redness or ulcers.

In some further embodiments, the present invention provides methods of treating or inhibiting radiation cystitis in a subject suspected of being exposed to a cytotoxic agent or to radiation, the method comprising providing to said subject an effective amount of an ERβ selective ligand.

In some further embodiments, the present invention provides methods of treating or inhibiting mucositis in a subject suspected of being exposed to a cytotoxic agent or to radiation, the method comprising providing to said subject an effective amount of an ERβ selective ligand.

In some further embodiments, the present invention provides methods of treating or inhibiting mucositis in a subject in need thereof, said mucositis induced by exposure to a cytotoxic agent or to radiation, the method comprising administering to said subject escalating doses of an ERβ selective ligand.

In some of each of the foregoing embodiments, the ERβ selective ligand is applied topically. In some of each of the foregoing embodiments, the ERβ selective ligand is non-uterotrophic, non-mammotrophic, or non-uterotrophic and non-mammotrophic.

In some embodiments of the foregoing methods, the subject is a human. In some further embodiments of the foregoing methods, the exposure to a cytotoxic agent or to radiation is attendant to a therapeutic or diagnostic procedure. In some further embodiments of the foregoing methods, the exposure to a cytotoxic agent or to radiation is accidental. In some further embodiments of the foregoing methods, the exposure to a cytotoxic agent or to radiation is as a result of an industrial accident or a terrorist incident.

In some further embodiments of the foregoing methods, methods further comprise the administration of an effective amount of at least one traditional medicament. In some such embodiments, the traditional treatment is administered to the subject contemporaneously with the non-uterotropic, non-mammotrophic ERβ selective ligand.

In some embodiments of the foregoing methods, the binding affinity of the ERβ selective ligand to ERβ is at least about 20 times greater than its binding affinity to ERα. In further embodiments, the binding affinity of the ERβ selective ligand to ERβ is at least about 50 times greater than its binding affinity to ERα.

In some further embodiments of the foregoing methods, the ERβ selective ligand causes an increase in wet uterine weight is less than about 25% of that observed for a maximally efficacious dose of 17β-estradiol in a standard pharmacological test procedure measuring uterotrophic activity, for example the uterotrophic test procedure as described herein.

In some further embodiments of the foregoing methods, the ERβ selective ligand causes an increase in defensin β1 mRNA which is less than about 25% of that observed for a maximally efficacious dose of 17β-estradiol in a standard pharmacological test procedure measuring mammotrophic activity, for example, the Mammary End Bud Test Procedure as described herein.

In some further embodiments of the foregoing methods, the ERβ selective ligand causes an increase in wet uterine weight which is less than about 10% of that observed for a maximally efficacious dose of 17β-estradiol in a standard pharmacological test procedure measuring uterotrophic activity. In some further embodiments, the ERβ selective ligand causes an increase in defensin β1 mRNA which is less than about 10% of that observed for a maximally efficacious dose of 17β-estradiol in a standard pharmacological test procedure measuring mammotrophic activity. In some embodiments, defensin β1 mRNA is detected using one or more of SEQ ID NO:1, SEQ ID NO:2 or SEQ ID NO:3.

In some further embodiments of the foregoing methods, the ERβ selective ligand does not significantly (p>0.05) increase wet uterine weight compared with a control that is devoid of uterotrophic activity, and does not significantly (p>0.05) increase defensin β1 mRNA compared with a control that is devoid of mammotrophic activity.

In some embodiments of the foregoing methods, the ERβ selective ligand has the Formula I: 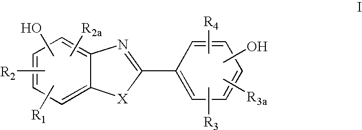
wherein:

R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₅, R₆ are each, independently, hydrogen, alkyl of 1-6 carbon atoms, aryl of 6-10 carbon atoms;

X is O, S, or NR₇;

R₇ is hydrogen, alkyl of 1-6 carbon atoms, aryl of 6-10 carbon atoms, —COR₅, —CO₂R₅ or —SO₂R₅;

or a pharmaceutically acceptable salt thereof. In some such embodiments, the ERβ selective ligand has the Formula II: 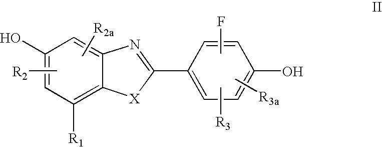
wherein:

R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₅, R₆ are each, independently, hydrogen, alkyl of 1-6 carbon atoms, aryl of 6-10 carbon atoms;

X is O, S, or NR₇;

R₇ is hydrogen, alkyl of 1-6 carbon atoms, aryl of 6-10 carbon atoms, —COR₅, —CO₂R₅ or —SO₂R₅;

or a pharmaceutically acceptable salt thereof. In some such embodiments, X is O, and R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In some preferred embodiments of the foregoing methods, the ERβ selective ligand is a compound having the formula: 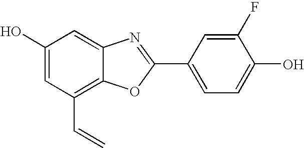
or a pharmaceutically acceptable salt thereof.

In some further embodiments of the foregoing methods, the ERβ selective ligand has the Formula II: 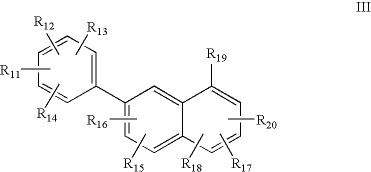
wherein:

R₁₁, R₁₂, R₁₃, and R₁₄ are each, independently, selected from hydrogen, hydroxyl, alkyl of 1-6 carbon atoms, alkoxy of 1-6 carbon atoms, or halogen;

R₁₅, R₁₆, R₁₇, R₁₈, R₁₉, and R₂₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₅, R₁₆, R₁₇, R₁₆, R₁₉, or R₂₀ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;

wherein at least one of R₁₁, R₁₂, R₁₃, R₁₄, R₁₇, R₁₈, R₁₉ or R₂₀ is hydroxyl, or a pharmaceutically acceptable salt thereof. In some such embodiments, the ERβ selective ligand has the Formula IV: 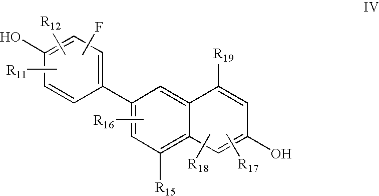
wherein:

R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;

R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;

wherein at least one of R₁₅ or R₁₉ is not hydrogen, or a pharmaceutically acceptable salt thereof. In some such embodiments, the ERβ selective ligand has the Formula V: 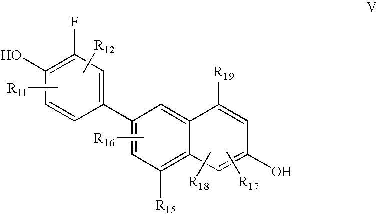
wherein:

R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;

R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈ or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;

wherein at least one of R₁₅ or R₁₉ is not hydrogen, or a pharmaceutically acceptable salt thereof. In some further embodiments, the ERβ selective ligand has the Formula V, wherein the 5 or 6-membered heterocyclic ring having 1 to 4 heteroatoms selected from O, N or S is furan, thiophene or pyridine, and R₁₅, R₁₆, R₁₇, R₁₈, and R₁₉ are each, independently, hydrogen, halogen, —CN, or alkynyl of 2-7 carbon atoms. In some such embodiments, R₁₆, R₁₇, and R₁₈ are hydrogen. In some further embodiments of the foregoing methods, the ERβ selective ligand is a compound having the formula: 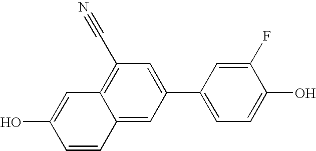
or a pharmaceutically acceptable salt thereof.

The preparation of compounds of Formulas III, IV and V is described in US Published Application 2003/0181519, U.S. Pat. No. 6,914,074, and PCT US 02/39883, filed Dec. 2, 2002, each of which is incorporated by reference herein in its entirety.

In some further embodiments of the foregoing methods, the ERβ selective ligand has the Formula VII: 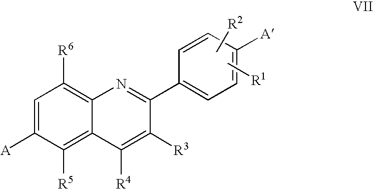
wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C₆ alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, or heteroaryl;
R⁵ and R⁶ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H;
   or a N-oxide thereof.

In some further embodiments of the foregoing methods, the ERβ selective ligand has the Formula X: 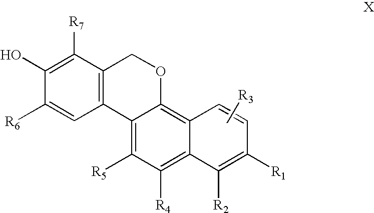
wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁, or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl; wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
   or a pharmaceutically acceptable salt or prodrug thereof.

In some further embodiments of the foregoing methods, the ERβ selective ligand is a compound having the formula:

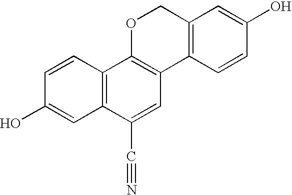

The preparation of ERβ selective ligands having Formula VII is described in U.S. patent application Ser. No. 10/846,216, US Published Application US 2005/0009784, published Jan. 13, 2005, and WO 04/103973. The preparation of ERβ selective ligands having Formula X is disclosed in US Published Application US2003/0176941, published Sep. 18, 2003, U.S. Pat. No. 6,723,747, and PCT US 02/39802, filed Dec. 12, 2002. Each of the foregoing patents and applications is incorporated herein by reference in its entirety.

The present invention also provides compositions comprising a therapeutically effective amount of an ERβ selective ligand, and a traditional mediation for mucositis or cystitis. In some embodiments, the ERβ selective ligand is applied topically. In some further embodiments, the ERβ selective ligand is non-uterotrophic, non-mammotrophic, or non-uterotrophic and non-mammotrophic.

Therapeutic Methods

Methods of Treating or Inhibiting Mucositis

The present invention provides methods of treating or inhibiting mucositis in a subject in need thereof wherein the mucositis is induced by exposure to a cytotoxic agent or to radiation. The method comprises providing to the subject an effective amount of one or more, preferably one, ERβ selective ligands. In some embodiments, the ERβ selective ligand is applied topically. In some further embodiments, the ERβ selective ligand is non-uterotrophic, non-mammotrophic, or non-uterotrophic and non-mammotrophic. In some embodiments the subject is a human.

As used herein the terms “treatment”, “treating”, “treat” and the like are refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a human, and includes: (a) preventing the disease or symptom from occurring in a subject which may be predisposed to the disease or symptom but has not yet been diagnosed as having it; (b) inhibiting one or more disease symptom, i.e., arresting its development; or relieving the disease symptom, i.e., causing regression of the disease or symptom.

The terms “individual”, “subject”, “host” and “patient” are used interchangeably and refer to any subject for whom diagnosis, treatment, or therapy is desired, particularly humans. Other subjects may include cattle, dogs, cats, guinea pigs, rabbits, rats, mice, horses, and the like. In some preferred embodiments the subject is a human.

As used herein, the term “mucositis” refers to inflammation of any mucous membrane. It encompasses terms such as stomatitis, esophagitis and proctitis. In some embodiments the mucositis is caused by exposure to radiation or to one or more cytotoxic agents. The exposure may be secondary to cancer treatments or in preparation for hematopoetic stem cell transplantation. Other causes of mucositis include accidental or malicious exposure to radiation or cytotoxic agents. In some embodiments the mucositis is oral mucositis, gastrointestinal mucositis, or rectal mucositis.

As used herein, the terms “administering” or “providing” mean either directly administering the ERβ selective ligand, or administering a prodrug, derivative, or analog of the ERβ selective ligand that will form an effective amount of the ERβ selective ligand within the body. The terms include routes of administration that are systemic (e.g., via injection, orally in a tablet, pill, capsule, or other dosage form useful for systemic administration of pharmaceuticals, and the like, such as described herein below), and topical (e.g., creams, solutions, and the like, including solutions such as mouthwashes, for topical oral administration).

The term “in need thereof” and the like as used herein refers to an subject that has been determined to be in need of treatment for a disease such as, for example, mucositis or cystitis, preferably mucositis or cystitis induced by exposure or suspected exposure to radiation or a cytotoxic agent, or for a symptom of mucositis, cystitis or a symptom of exposure to radiation or a cytotoxic agent. Such a determination may be a result of a medical diagnosis. Further, subjects “in need” of the methods of the present invention include those known or suspected to have been exposed to radiation or cytotoxic agents. Other subjects “in need” of the methods of the present invention include those at increased risk of exposure to radiation and/or cytotoxic agents. Examples of these subjects include without limitation those actively being treated with radiation and/or chemotherapeutics, those who routinely come into contact with radiation or cytotoxic agents (e.g. medical workers, those involved in the manufacture and/or distribution of chemotherapeutics, or those in the nuclear industry), for example.

As used herein, the phrase “exposed to radiation” and the like refers to any exposure, intended or unintended, to radiation. Radiation may be of any type including α-, β-, and Γ-radiation.

As used herein, the term “cytotoxic agent” refers to a composition which causes cell death in a subject. In some embodiments the cytotoxic agent is a chemotherapeutic agent.

ERβ selective ligands are known to those of skilled in the art as compounds which preferentially bind to ERβ. The preparation of certain exemplary ERβ selective ligands, including those of Formulas I and II, such as 2-(3-fluoro-4-hydroxyphenyl)-7-vinyl-1,3-benzoxazol-5-ol (ERB-041), is described in U.S. Pat. No. 6,794,403, and WO 03/050095, each of which is incorporated herein by reference in its entirety. In some embodiments, ERβ selective ligands include compounds set forth in U.S. Pat. No. 6,794,403, WO 03/050095, U.S. patent application Ser. No. 10/316,640, filed Dec. 11, 2002 and published as US 20030181519 on Sep. 25, 2003; U.S. Patent Application Ser. No. 60/637,144, filed Dec. 17, 2004, and PCT application no. US2005/045375, each of which is incorporated herein by reference in its entirety.

In some embodiments, the ERβ selective ligand is 2-(3-fluoro-4-hydroxyphenyl)-7-vinyl-1,3-benzoxazol-5-ol, which has the Formula:

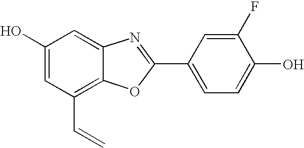

In some embodiments, the ERβ selective ligand is 3-(3-fluoro-4-hydroxyphenyl)-7-hydroxy-1-naphthonitrile, which has the Formula:

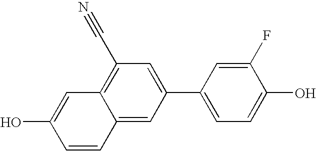

As used herein, the term “ERβ selective ligand” means that the binding affinity (as measured by IC₅₀, where the IC₅₀ of 17β-estradiol is not more than 3 fold different between ERα and ERβ) of the ligand to ERβ is at least about 10 times greater than its binding affinity to ERα in a standard pharmacological test procedure that measures the binding affinities to ERβ and ERα. It is preferred that the ERβ selective ligand will have a binding affinity to ERβ that is at least about 20 times greater than its binding affinity to ERα. It is more preferred that the ERβ selective ligand will have a binding affinity to ERβ that is at least about 50 times greater than its binding affinity to ERα. It is further preferred that the ERβ selective ligand is non-uterotrophic and non-mammotrophic.

As used in accordance with this invention, the term “non-uterotrophic” means producing an increase in wet uterine weight in a standard pharmacological test procedure of less than about 50% of the uterine weight increase observed for a maximally efficacious dose of a positive control in the same procedure. In some preferred embodiments the standard pharmacological test procedure measuring uterotrophic activity is the pharmacological test procedure published in Harris H A, et al, Endocrinology 2002; 143(11):4172-4177, referred to hereinafter as the “uterotrophic test procedure”. In some embodiments the positive control is 17β-estradiol, 17α-ethinyl-17β-estradiol or diethylstilbestrol (DES). It is preferred that the increase in wet uterine weight will be less than about 25% of that observed for the positive control, and more preferred that the increase in wet uterine weight will be less than about 10% of that observed for the positive control. It is most preferred that the non-uterotrophic ERβ selective ligand will not significantly increase wet uterine weight (p>0.05), as determined by analysis of variance using a least significant difference test, compared with a control that is devoid of uterotrophic activity (e.g. vehicle). The maximally efficacious dose of the positive control will vary depending on a number of factors including but not limited to the specific assay methodology, the identity of the positive control, amount and identity of vehicle, etc. In some embodiments, the positive control is 17β-estradiol and the maximally efficacious dose is between 0.1 μg/kg and 100 μg/kg, preferably between 1.0 μg/kg and 30 μg/kg; more preferably between 3 μg/kg and 30 μg/kg; and more preferably between 10 μg/kg and 20 μg/kg. In some embodiments, the positive control is 17α-ethinyl-17β-estradiol and the maximally efficacious dose is between 0.1 μg/kg and 100 μg/kg, preferably between 1.0 μg/kg and 30 μg/kg; more preferably between 3 μg/kg and 30 μg/kg; and more preferably between 10 μg/kg and 20 μg/kg. In some embodiments, the positive control is DES and the maximally efficacious dose is between 0.1 μg/kg and 100 μg/kg, preferably between 1.0 μg/kg and 30 μg/kg; more preferably between 3 μg/kg and 30 μg/kg; and more preferably between 10 μg/kg and 20 μg/kg.

As used herein, the term “non-mammotrophic” means a compound that does not stimulate mammary gland development. In some embodiments, “non-mammotrophic” refers to producing an increase in defensin β1 mRNA in a standard pharmacological test procedure of less than about 50% of the defensin β1 mRNA increase observed for a maximally efficacious dose of 17β-estradiol (given in combination with progesterone) in the same procedure. In some embodiments, the standard pharmacological test procedure measuring mammotrophic activity is the Mammary End Bud Test Procedure. In some embodiments it is preferred that the increase defensin β1 mRNA will be less than about 25% of that observed for a positive control, and more preferred that the increase in defensin β1 mRNA will be less than about 10% of that observed for the positive control. It is most preferred that the non-mammotrophic ERβ selective ligand will not significantly increase defensin β1 mRNA (p>0.05) compared with a control that is devoid of mammotrophic activity (e.g. vehicle). In some embodiments, “non-mammotrophic” compounds can be identified using assays for measuring defensin β1 levels including, but not limited to, RT-PCR, Northern blots, in situ hybridization, immunohistochemistry (IHC), and Western blots. In some embodiments, compounds that are “non-mammotrophic” can be determined using histology, e.g., by confirming the absence of physical markers of mammary gland development. In some embodiments, indicators include without limitation, ductal elongation and appearance of lobulo-alveolar endbuds.

The present invention also provides methods of treating or inhibiting mucositis in a subject suspected of being exposed to a cytotoxic agent or to radiation. The methods comprise providing to the subject an effective amount of one or more non-uterotrophic, non-mammotrophic ERβ selective ligands.

In some embodiments, exposure to a cytotoxic agent or to radiation is attendant to a therapeutic or diagnostic procedure. In some embodiments, the exposure to a cytotoxic agent or to radiation is accidental. In some embodiments, the exposure to a cytotoxic agent or to radiation is as a result of a terrorist incident.

Methods of Treating or Inhibiting Radiation Cystitis

The present invention also provides methods of treating or inhibiting radiation cystitis in a subject. In some embodiments radiation cystitis induced by exposure to a cytotoxic agent or to radiation. The methods comprise providing to the subject an effective amount of one or more, preferably one, ERβ selective ligand. In some embodiments, the ERβ selective ligand is applied topically. In some further embodiments, the ERβ selective ligand is non-uterotrophic, non-mammotrophic, or non-uterotrophic and non-mammotrophic. In some embodiments the subject is a human.

The present invention also provides methods of treating or preventing symptoms of radiation cystitis in a subject suspected of being exposed to a cytotoxic agent or to radiation. The methods comprise providing to the subject an effective amount of one or more, preferably one, non-uterotropic, non-mammotrophic ERβ selective ligands. In some embodiments, the ERβ selective ligand is applied topically. In some further embodiments, the ERβ selective ligand is non-uterotrophic, non-mammotrophic, or non-uterotrophic and non-mammotrophic.

As used in accordance with this invention, the term “radiation cystitis” refers to inflammation of the bladder secondary to radiation exposure or exposure to a cytotoxic agent. The radiation exposure may be therapeutic (as for cancer therapy) or unintentional such as following accidental or malicious exposure (e.g. a nuclear accident, war or act of terrorism).

Methods of Ameliorating Symptoms of Mucositis or Cystitis

The present invention also provides methods for ameliorating symptoms of mucositis or cystitis by administering of an ERβ selective ligand to a subject. Several symptoms of mucositis and cystitis are discussed above. In some embodiments an effective amount of one or more, preferably one, ERβ selective ligands is administered to a subject in need thereof. In some embodiments, the ERβ selective ligand is applied topically. In some further embodiments, the ERβ selective ligand is non-uterotrophic, non-mammotrophic, or non-uterotrophic and non-mammotrophic.

In some embodiments, the methods of the present invention further comprise the administration of an effective amount of at least one traditional medicament. In some embodiments the traditional medicament is administered to the subject contemporaneously with the ERβ selective ligand.

Methods of Treating Symptoms of Exposure

The present invention further provides methods of treating at least one symptom of exposure of a subject to a cytotoxic agent or to radiation. The methods comprise providing to the subject an effective amount of an ERβ selective ligand. In some embodiments the symptom is selected from the group consisting of dysuria, haematuria, oedema, hyperaemia, petechiae, and ulceration of the bladder. In some embodiments the symptom is selected from the group consisting of redness, dryness, or swelling of the mouth, burning or discomfort when eating or drinking, open sores in the mouth and throat, abdominal cramps, rectal redness or ulcers. In some embodiments, the ERβ selective ligand is applied topically. In some further embodiments, the ERβ selective ligand is non-uterotrophic, non-mammotrophic, or non-uterotrophic and non-mammotrophic.

The present invention also provides methods of treating or preventing symptoms of exposure in a subject suspected of being exposed to a cytotoxic agent or to radiation. The methods comprise providing to the subject an effective amount of one or more, preferably one, ERβ selective ligands. In some embodiments, the ERβ selective ligand is applied topically. In some further embodiments, the ERβ selective ligand is non-uterotrophic, non-mammotrophic, or non-uterotrophic and non-mammotrophic.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. Alkyl groups can contain from 1 to about 20, 1 to about 10, 1 to about 8, 1 to about 6, 1 to about 4, or 1 to about 3 carbon atoms. In some embodiments, alkyl groups can be substituted with up to four substituent groups, as described below. As used herein, the term “lower alkyl” is intended to mean alkyl groups having up to six carbon atoms.

As used herein, “alkenyl” refers to an alkyl group having one or more double carbon-carbon bonds. Example alkenyl groups include ethenyl, propenyl, butenyl, pentenyl, hexenyl, butadienyl, pentadienyl, hexadienyl, and the like. In some embodiments, alkenyl groups can be substituted with up to four substituent groups, as described below.

As used herein, “alkynyl” refers to an alkyl group having one or more triple carbon-carbon bonds. Examples of alkynyl groups include ethynyl, propynyl, butynyl, pentynyl, and the like. In some embodiments, alkynyl groups can be substituted with up to four substituent groups, as described below.

As used herein, “cycloalkyl” refers to non-aromatic carbocyclic groups including cyclized alkyl, alkenyl, and alkynyl groups. Cycloalkyl groups can be monocyclic (e.g., cyclohexyl) or poly-cyclic (e.g. 2, 3, or 4 fused ring, bridged, or spiro monovalent saturated hydrocarbon moiety), wherein the carbon atoms are located inside or outside of the ring system. Any suitable ring position of the cycloalkyl moiety may be covalently linked to the defined chemical structure. Examples of cycloalkyl groups include cyclopropyl, cyclopropylmethyl, cyclobutyl, cyclopentyl, cyclohexyl, cyclohexylmethyl, cyclohexylethyl, cycloheptyl, cyclopentenyl, cyclohexenyl, cyclohexadienyl, cycloheptatrienyl, norbornyl, norpinyl, norcarnyl, adamantyl, spiro[4.5]deanyl, homologs, isomers, and the like. Also included in the definition of cycloalkyl are moieties that have one or more aromatic rings fused (i.e., having a bond in common with) to the cycloalkyl ring, for example, benzo derivatives of cyclopentane (indanyl), cyclohexane (tetrahydronaphthyl), and the like.

As used herein, “hydroxy” or “hydroxyl” refers to OH.

As used herein, “halo” or “halogen” includes fluoro, chloro, bromo, and iodo.

As used herein, “cyano” refers to CN.

As used herein, “alkoxy” refers to an —O-alkyl group. Example alkoxy groups include methoxy, ethoxy, propoxy (e.g., n-propoxy and isopropoxy), t-butoxy, and the like. An alkoxy group can contain from 1 to about 20, 1 to about 10, 1 to about 8, 1 to about 6, 1 to about 4, or 1 to about 3 carbon atoms. In some embodiments, alkoxy groups can be substituted with up to four substituent groups, as described below.

As used herein, the term “perfluoroalkoxy” indicates a group of formula —O-perfluoroalkyl.

As used herein, “haloalkyl” refers to an alkyl group having one or more halogen substituents. Examples of haloalkyl groups include CF₃, C₂F₅, CHF₂, CCl₃, CHCl₂, C₂Cl₅, and the like. An alkyl group in which all of the hydrogen atoms are replaced with halogen atoms can be referred to as “perhaloalkyl.” Examples perhaloalkyl groups include CF₃ and C₂F₅.

As used herein, “haloalkoxy” refers to an —O-haloalkyl group.

As used herein, “aryl” refers to aromatic carbocyclic groups including monocyclic or polycyclic aromatic hydrocarbons such as, for example, phenyl, 1-naphthyl, 2-naphthyl anthracenyl, phenanthrenyl, and the like. In some embodiments, aryl groups have from 6 to about 20 carbon atoms.

As used herein, “heterocyclic ring” is intended to refer to a monocyclic aromatic or non-aromatic ring system having from 5 to 10 ring atoms and containing 1-3 hetero ring atoms selected from O, N and S. In some embodiments, one or more ring nitrogen atoms can bear a substituent as described herein.

As used herein, “arylalkyl” or “aralkyl” refers to a group of formula -alkyl-aryl. Preferably, the alkyl portion of the arylalkyl group is a lower alkyl group, i.e., a C₁₋₈ alkyl group, more preferably a C₁₋₃ alkyl group. Examples of aralkyl groups include benzyl and naphthylmethyl groups.

At various places in the present specification substituents of compounds of the invention are disclosed in groups or in ranges. It is specifically intended that the invention include each and every individual subcombination of the members of such groups and ranges. For example, the term “C₁ alkyl” is specifically intended to individually disclose methyl, ethyl, propyl, isopropyl, n-butyl, sec-butyl, isobutyl, etc.

Administration and Pharmaceutical Compositions

The ERβ selective ligand agonist may be administered alone or may be delivered in a mixture with other drugs, such as those disclosed above, for treating cystitis, mucositis, or other disease, symptom or condition associated with cystitis or mucositis or attendant to exposure or suspected exposure to a cytotoxic agent or radiation. In some embodiments, a common administration vehicle (e.g., pill, tablet, implant, injectable solution, etc.) would contain both an ERβ selective ligand and additional therapeutic agent(s). Thus, the present invention also provides pharmaceutical compositions, for medical use, which comprise the ERβ selective ligand of the invention together with one or more pharmaceutically acceptable carriers thereof and optionally other therapeutic ingredients.

In accordance with the present invention, treatment can also include combination therapy. As used herein “combination therapy” means that the patient in need of treatment is treated or given another drug or treatment modality for the disease in conjunction with the ERβ selective ligand of the present invention. This combination therapy can be sequential therapy where the patient is treated first with one and then the other, or the two or more treatment modalities are given simultaneously. Preferably, the treatment modalities administered in combination with the ERβ selective ligands do not interfere with the therapeutic activity of the ERβ selective ligand.

In some embodiments, administration of an ERβ selective ligand can be combined with traditional mucositis or cystitis treatments, e.g. combined with a “traditional treatment”. Preferably, the traditional treatment does not interfere with or reduce the effectiveness of the ERβ selective ligand. The traditional treatment may or may not include non-drug based treatments.

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. It is projected that effective administration of the compounds of this invention may be given at a daily oral dose of from about 5 μg/kg to about 100 mg/kg. The projected daily dosages are expected to vary with route of administration, and the nature of the compound administered. In some embodiments the methods of the present invention comprise administering to the subject escalating doses of an ERβ selective ligand. In some embodiments, the ERβ selective ligand is applied topically. In some further embodiments, the ERβ selective ligand is non-uterotrophic, non-mammotrophic, or non-uterotrophic and non-mammotrophic.

Such doses may be administered in any manner useful in directing the active compounds herein to the recipient's bloodstream, including orally, via implants, parenterally (including intravenous, intraperitoneal and subcutaneous injections), intraarticularly, rectally, intranasally, intraocularly, vaginally, or transdermally.

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time-release formulations to alter the absorption of the active compound(s). The oral formulation may also consist of administering the active ingredient in water or a fruit juice, containing appropriate solubilizers or emulsifiers as needed.

In some cases it may be desirable to administer the compounds directly to the airways in the form of an aerosol.

The compounds of this invention may also be administered parenterally (such as directly into the joint space) or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

In some embodiments, the methods of the invention are performed via topical administration of the ERβ selective ligand. In some such embodiments, the topical administration is via a mouthwash solution, for example as described in the oral mucositis test procedure, discussed below.

Additional numerous various excipients, dosage forms, dispersing agents and the like that are suitable for use in connection with the solid dispersions of the invention are known in the art and described in, for example, Remington's Pharmaceutical Sciences, 17th ed., Mack Publishing Company, Easton, Pa., 1985, which is incorporated herein by reference in its entirety.

Kits

In some embodiments, a kit comprising one or more ERβ selective ligands useful for the treatment of the diseases or disorders described herein is provided. The kit comprises a container and a label or package insert on or associated with the container. Suitable containers include, for example, bottles, vials, syringes, etc. The containers can be formed from a variety of materials such as glass or plastic. The container holds or contains a composition that is effective for treating the disease or disorder of choice and may have a sterile access port (for example the container may be an intravenous solution bag or a vial having a stopper pierceable by a hypodermic injection needle). At least one active agent in the composition is an ERβ selective ligand. The label or package insert indicates that the composition is used for treating a patient having or predisposed to mucositis or cystitis or for a patient exposed to or thought to have been exposed to radiation and/or a cytotoxic agent. The article of manufacture can further include a second container having a pharmaceutically acceptable diluent buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. Optionally the kit may contain other components including, without limitations, sensors for detecting exposure to radiation and/or a cytotoxic agent, positive and negative controls, or traditional medicaments for the treatment of cystitis or mucositis. ERβ selective ligands can be tested using a number of methods known to those skilled in the art. Such methods include, for example, measuring relative binding affinities to ERβ and ERα and assessing on ore more activities in well-known assays.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Evaluation of Binding Affinities to ERβ and ERα

Compounds can be evaluated for their ability to compete with 17β-estradiol using both ERβ and ERα. This test procedure provides the methodology for one to determine the relative binding affinities for the ERβ or ERα. The procedure used is as described in Harris H A, et al, Steroids 2002; 67(5):379-384.

### Example 2

### Evaluation of Uterotrophic Activity

Uterotrophic activity of a test compound can be measured according to the standard pharmacological test procedure as published in Harris H A, et al, Endocrinology 2002; 143(11):4172-4177. For the sake of brevity, the standard pharmacological test procedure as published in Harris et al. will be referred to as the “uterotrophic test procedure”.

### Example 3

### Evaluation in the Mammary End Bud Test Procedure

Estrogens are required for full ductal elongation and branching of the mammary ducts, and the subsequent development of lobulo-alveolar end buds under the influence of progesterone. In this test procedure, the mammotrophic activity of ERβ selective compounds can be evaluated as follows. Seven week old C57/bl6 mice (Taconic Farms, Germantown, N.Y.) are ovariectomized and rested for about nine days. Animals are housed under a 12-hour light/dark cycle and fed a casein-based Purina Laboratory Rodent Diet 5K96 (Purina, Richmond, Ind.) and water ad libidtum. Mice are then dosed for seven days with vehicle, 17β-estradiol (1 μg/kg, subcutaneously in a vehicle of 50% DMSO/50% 1× Dulbecco's phosphate buffered saline) or an ERβ selective ligand (various doses, orally in a vehicle of 2% Tween-80/0.5% methylcellulose). For the final four days, mice are also dosed subcutaneously with progesterone (30 mg/kg, subcutaneously in a vehicle of 50% DMSO/50% 1× Dulbecco's phosphate buffered saline). On the seventh day, mice are euthanized and the number 4 or 9 inguinal mammary gland and underlying fat pad are excised. The fat pad is analyzed for defensin 1β mRNA expression as a marker of end bud proliferation. Total RNA is prepared individually from each mammary gland. Each sample is homogenized in 2 mLs of QIAzol lysis reagent (Qiagen; Valencia, Calif.) for 15-25 seconds using a Polytron homogenizer PT3100 (Brinkmann; Westbury, N.Y.). After 1 mL of this homogenate is extracted with 0.2 mL of chloroform and centrifuged at 4° C. for 15 minutes, about 0.5 mL aqueous phase is collected. The RNA from the aqueous phase is then purified using. Qiagen RNeasy kits according to the manufacturer's protocol. The trace genomic DNA in RNA sample is removed by on column RNase-Free DNase treatment during RNA purification. The RNA concentration is adjusted to 0.05 mg/ml for assay. Messenger RNA expression is analyzed using real-time quantitative-PCR on an ABI PRISM 7700 Sequence Detection System according to the manufacturer's protocol (Applied Biosystems Inc; Foster City Calif.).

Defensin β1 sequences are known to the art skilled and include, for example, GenBank accession numbers BC024380 (mouse) and NM_{—}005218 (human). The sequences of primers and labeled probes used for defensin β1 mRNA detection are as follows: forward primer, 5′-AATGCCTTCAACATGGAGGATT-3 (SEQ ID NO:1); reverse primer, 5′-TTACAGGTTCCCTGTAGTTTGGTATTAG-3′ (SEQ ID NO:2); probe, 5′FAM-TGTCTCCGCTCCAGCTGCCCA-TAMRA-3′ (SEQ ID NO:3). To compare mRNA expression levels between samples, defensin β1 mRNA expression is normalized to 18S RNA expression using primers and labeled probes from an Applied Biosystems TaqMan ribosomal RNA control reagent kit (VIC probe) for 18S mRNA detection. The expected result is that defensin β1 mRNA will be strongly upregulated by the combination of 17β-estradiol and progesterone, but not by either compound given alone. Test compounds, then, are evaluated for their ability to substitute for 17β-estradiol in this regimen.

### Example 4

### Evaluation in the Oral Mucositis Test Procedure

This standard pharmacological test procedure, which induces oral mucositis in the hamster cheek pouch, is described in Sonis S T et al (Cytokine 1997; 9(8):605-612).

### Example 5

### Evaluation in the Test Procedure of Methotrexate-Induced Intestinal Mucositis

This standard pharmacological test procedure, which uses methotrexate to induce intestinal mucositis is described in Carneiro B A et al (Digestive Diseases & Sciences 2004; 49(1):65-72).

### Example 6

### Evaluation in the Combined Chemotherapy- and Radiation-Induced Test Procedure of Mucositis

This standard pharmacological test procedure is described in Orazi A et al (Lab Invest. 1996 July; 75(1):33-42).

### Example 7

### Evaluation in the Radiation-Induced Cystitis Test Procedure

This standard pharmacological test procedure is described in Kanai A, Z et al. (American Journal of Physiology Renal Physiology 2002; 283:F1304-F1312).

### Example 8

### Evaluation of Estrogen Receptor-β Selective Agonist in Hamsters

Acute Radiation Model of Oral Mucositis

The acute radiation model in hamsters has proven to be an accurate, efficient and cost-effective technique to provide a preliminary evaluation of anti-mucositis compounds (Sonis et al., Oral Surg Oral Med Oral Pathol 1990; 69(4):437-448). The course of mucositis in this model is well defined and results in peak scores approximately 14-16 Days following radiation. The acute model has little systemic toxicity, resulting in few hamster deaths which makes the acute model amenable for initial efficacy studies. The acute model has also been used to study specific mechanistic elements in the pathogenesis of mucositis.

Mucositis Evaluation

Forty (40) Male LVG Syrian Golden Hamsters (Charles River Laboratories), aged 5 to 6 weeks, with average body weight of 116.3 g at study commencement, were used. Animals were individually numbered using an ear punch and housed 10 animals per cage. Animals were fed with a Purina Labdiet® 5061 rodent diet and water was provided ad libitum. Animals were acclimatized for two days prior to study commencement.

Animals were randomly and prospectively divided into four (4) treatment groups prior to irradiation. Mucositis was induced using a standardized acute radiation protocol, where a single dose of radiation (40 Gy/dose) was administered to all animals on day 0. Radiation was generated with a 160 kilovolt potential (15-ma) source at a focal distance of 30 cm, hardened with an AI filtration system. Irradiation targeted the left buccal pouch mucosa at a rate of 3.2 Gy/minute. Prior to irradiation, animals were anesthetized with an intra-peritoneal injection of Ketamine (160 mg/kg) and Xylazine (8 mg/kg). The left buccal pouch was everted, fixed and isolated using a lead shield.

Controls or 3-(3-Fluoro-4-hydroxy-phenyl)-7-hydroxy-naphthalene-1-carbonitrile (Compound 1) were given in the volumes and by the routes described in Table 1 and dosing began the day before radiation treatment.

TABLE 1
Study Design

| Group | Number of | Cancer | | Treatment | Vol. |
|---|---|---|---|---|---|
| Number | Animals | Therapy | Treatment | Schedule* | (mL) |
| 1 | 10 males | Radiation only | Vehicle control | Once daily, | 0.2 |
| | | | (2% tween-80/0.5% | beginning on day -1 | |
| | | | methylcellulose) | | |
| | | | Gavage | | |
| 2 | 10 males | Radiation only | Compound 1 (10 mg/kg) | Once daily, | 0.2 |
| | | | Gavage | beginning on day -1 | |
| 3 | 10 males | Radiation only | Vehicle control | Once daily, | 0.5 |
| | | | (0.00021% | beginning on day -1 | |
| | | | hydroxypropyl-beta- | | |
| | | | cyclodextran, pH ˜8.3) | | |
| | | | Topical (intrapouch) | | |
| 4 | 10 males | Radiation only | Compound 1 (10 mg/kg) | Once daily, | 0.5 |
| | | | Topical (intrapouch) | beginning on day -1 | |

The grade of mucositis was scored, beginning day 6, and for every second day thereafter, through and including day 28. For the evaluation of mucositis, the animals were anesthetized with an inhalation anesthetic, and the left pouch everted. Mucositis was scored visually by comparison to a validated photographic scale, ranging from 0 for normal, to 5 for severe ulceration (clinical scoring). In descriptive terms, this scale is defined as follows:
Score: Description:
0 Pouch completely healthy. No erythema or vasodilation.
1 Light to severe erythema and vasodilation. No erosion of mucosa.
2 Severe erythema and vasodilation. Erosion of superficial aspects of mucosa leaving denuded areas. Decreased stippling of mucosa.
3 Formation of off-white ulcers in one or more places. Ulcers may have a yellow/gray appearance due to pseudomembrane. Cumulative size of ulcers should equal about ¼ of the pouch. Severe erythema and vasodilation.
4 Cumulative seize of ulcers should equal about ½ of the pouch. Loss of pliability. Severe erythema and vasodilation.
5 Virtually all of pouch is ulcerated. Loss of pliability (pouch can only partially be extracted from mouth)

A score of 1-2 is considered to represent a mild stage of the disease, whereas a score of 3-5 is considered to indicate moderate to severe mucositis.

The effect on mucositis of each route of administration of Compound 1 compared to vehicle was assessed by determining the difference in the number of days hamsters in each group have ulcerative (score ≧3) mucositis. Statistical significance was assessed by a Chi-squared test and p<0.05 was considered statistically significant.

Results

Experimental results are set forth below in Table 2. When Compound 1 was administered by gavage, there was no significant change in the number of days hamsters in the two groups had ulcerative (score ≧3) mucositis. However, it is possible that variation in the dosage would produce a statistically significant effect.

When Compound 1 was administered topically (into the pouch), the number of days animals experienced ulcerative (score >3) mucositis was significantly reduced.

TABLE 2
Effect of Compound 1 on radiation-induced mucositis

| | | | Days with | % Days with | |
|---|---|---|---|---|---|
| | Route | | mucositis score | mucositis | |
| Treatment | of administration | Total Days | ≧3 | score ≧3 | P value |
| Vehicle | Topical | 240 | 118 | 49 | |
| Compound 1 | Topical | 240 | 72 | 30 | <0.001 |
| (500 nM) | | | | | |
| Vehicle | Gavage | 240 | 100 | 42 | |
| Compound 1 | Gavage | 240 | 106 | 44 | 0.645 |
| (10 mg/kg) | | | | | |

The materials, methods, and examples presented herein are intended to be illustrative, and are not intended to limit the scope of the invention. All publications, including patent applications, patents, Genbank accession records and other references mentioned herein are incorporated by reference in their entirety.

What is claimed is:

1. A method of treating or inhibiting mucositis in a subject in need thereof, said mucositis induced by exposure to a cytotoxic agent or to radiation, the method comprising administering to said subject an effective amount an ERβ selective ligand.

2. The method of claim 1, wherein the ERβ selective ligand is administered topically.

3. The method of claim 1, wherein the ERβ selective ligand is non-uterotrophic and non-mammotrophic.

4. The method of claim 1, wherein the binding affinity of the ERβ selective ligand to ERβ is at least about 20 times greater than its binding affinity to ERα.

5. The method of claim 1, wherein the mucositis is oral mucositis, gastrointestinal mucositis, or rectal mucositis.

6. A method of treating or inhibiting radiation cystitis in a subject, said radiation cystitis induced by exposure to a cytotoxic agent or to radiation, the method comprising administering to said subject an effective amount of an ERβ selective ligand.

7. The method of claim 6, wherein the ERβ selective ligand is administered topically.

8. The method of claim 6, wherein the ERβ selective ligand is non-uterotrophic and non-mammotrophic.

9. The method of claim 6, wherein the binding affinity of the ERβ selective ligand to ERβ is at least about 20 times greater than its binding affinity to ERα.

10. The method of claim 1, wherein the subject is a human.

11. The method of claim 6, wherein the subject is a human.

12. The method of claim 1, wherein the exposure to a cytotoxic agent or to radiation is attendant to a therapeutic or diagnostic procedure, or is accidental, or is as a result of an industrial accident or a terrorist incident.

13. The method of claim 6, wherein the exposure to a cytotoxic agent or to radiation is attendant to a therapeutic or diagnostic procedure, or is accidental, or is as a result of an industrial accident or a terrorist incident.

14. A method of treating at least one symptom of exposure of a subject to a cytotoxic agent or to radiation, the method comprising administering to said subject an effective amount of an ERβ selective ligand.

15. The method of claim 14, wherein the ERβ selective ligand is administered topically.

16. The method of claim 14, wherein the ERβ selective ligand is non-uterotrophic and non-mammotrophic.

17. The method of claim 14, wherein the at least one symptom is selected from the group consisting of dysuria, haematuria, oedema, hyperaemia, petechiae, and ulceration of the bladder.

18. The method of claim 14, wherein the at least one symptom is selected from the group consisting of redness, dryness, or swelling of the mouth, burning or discomfort when eating or drinking, open sores in the mouth and throat, abdominal cramps, rectal redness or ulcers.

19. The method of claim 14, further comprising the administration of an effective amount of at least one traditional medicament.

20. The method of claim 19, wherein the traditional treatment is administered to the subject contemporaneously with the ERβ selective ligand.

21. A method of treating or inhibiting radiation cystitis in a subject suspected of being exposed to a cytotoxic agent or to radiation, the method comprising administering to said subject an effective amount of an ERβ selective ligand.

22. The method of claim 21, wherein the ERβ selective ligand is administered topically.

23. The method of claim 21, wherein the ERβ selective ligand is non-uterotrophic and non-mammotrophic.

24. A method of treating or inhibiting mucositis in a subject suspected of being exposed to a cytotoxic agent or to radiation, the method comprising administering to said subject an effective amount of an ERβ selective ligand.

25. The method of claim 24, wherein the ERβ selective ligand is administered topically.

26. The method of claim 24, wherein the ERβ selective ligand is non-uterotrophic and non-mammotrophic.

27. The method of claim 14, wherein the exposure to a cytotoxic agent or to radiation is attendant to a therapeutic or diagnostic procedure, or is accidental, or is as a result of an industrial accident or a terrorist incident.

28. The method of claim 21, wherein the exposure to a cytotoxic agent or to radiation is attendant to a therapeutic or diagnostic procedure, or is accidental, or is as a result of an industrial accident or a terrorist incident.

29. The method of claim 24, wherein the exposure to a cytotoxic agent or to radiation is attendant to a therapeutic or diagnostic procedure, or is accidental, or is as a result of an industrial accident or a terrorist incident.

30. The method of claim 14, wherein the subject is a human.

31. The method of claim 21, wherein the subject is a human.

32. The method of claim 24, wherein the subject is a human.

33. A pharmaceutical composition comprising a therapeutically effective amount of an ERβ selective ligand, and a traditional mediation for mucositis or cystitis.

34. The method of claim 1, wherein said subject is administered escalating doses of said ERβ selective ligand.

35. The method of claim 6, wherein said subject is administered escalating doses of said ERβ selective ligand.

36. The method of claim 14, wherein said subject is administered escalating doses of said ERβ selective ligand.

37. The method of claim 21, wherein said subject is administered escalating doses of said ERβ selective ligand.

38. The method of claim 24, wherein said subject is administered escalating doses of said ERβ selective ligand.

39. The method of claim 1, wherein the ERβ selective ligand has the Formula I: 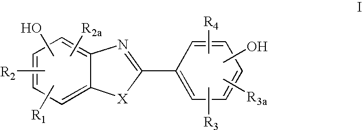
wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
or the Formula II: 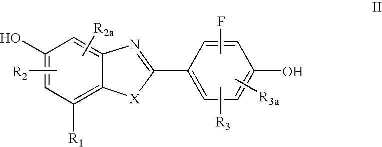
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
or
the Formula III: 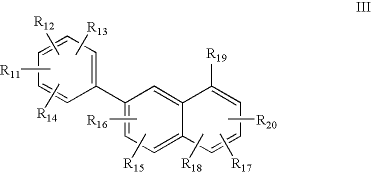
wherein:
R₁₁, R₁₂, R₁₃, and R₁₄ are each, independently, selected from hydrogen, hydroxyl, alkyl of 1-6 carbon atoms, alkoxy of 1-6 carbon atoms, or halogen;
R₁₅, R₁₆, R₁₇, R₁₈, R₁₉, and R₂₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, R₁₉, or R₂₀ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₁, R₁₂, R₁₃, R₁₄, R₁₇, R₁₈, R₁₉ or R₂₀ is hydroxyl, or a pharmaceutically acceptable salt thereof;
or
the Formula IV: 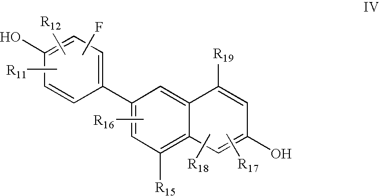
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen, or a pharmaceutically acceptable salt thereof;
or
the Formula V: 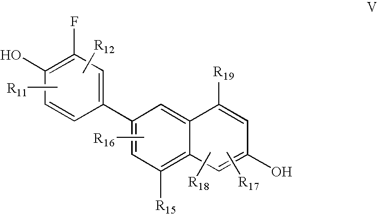
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈ or R₉ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen, or a pharmaceutically acceptable salt thereof;
or the Formula VII: 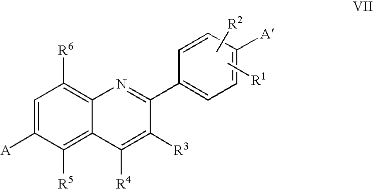
wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C₆ alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, or heteroaryl;
R⁵ and R⁶ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H;
or a N-oxide thereof;
or the Formula X: 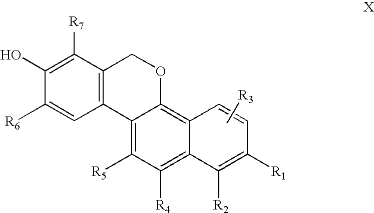
wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁, or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl; wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt or prodrug thereof.

40. The method of claim 1, wherein the ERβ selective ligand is a compound having the formula: 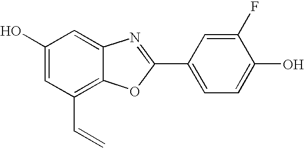
or a pharmaceutically acceptable salt thereof;
or the formula: 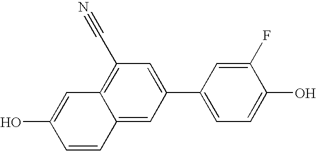
or a pharmaceutically acceptable salt thereof;
or the formula: 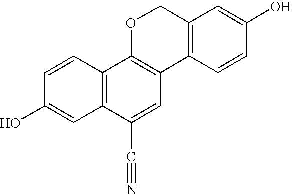
or a pharmaceutically acceptable salt thereof.

41. The method of claim 6, wherein the ERβ selective ligand has the Formula I: 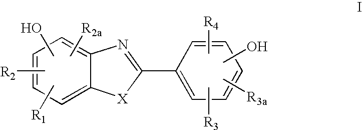
wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
or the Formula II: 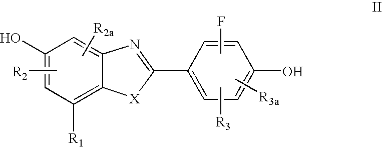
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
or
the Formula III: 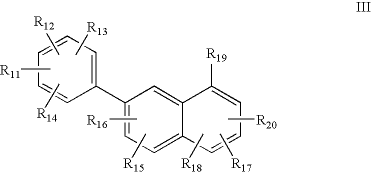
wherein:
R₁₁, R₁₂, R₁₃, and R₁₄ are each, independently, selected from hydrogen, hydroxyl, alkyl of 1-6 carbon atoms, alkoxy of 1-6 carbon atoms, or halogen;
R₁₅, R₁₆, R₁₇, R₁₈, R₁₉, and R₂₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, R₁₉, or R₂₀ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₁, R₁₂, R₁₃, R₁₄, R₁₇, R₁₈, R₁₉ or R₂₀ is hydroxyl, or a pharmaceutically acceptable salt thereof;
or
the Formula IV: 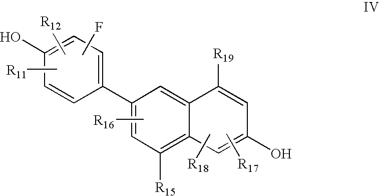
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen, or a pharmaceutically acceptable salt thereof;
or
the Formula V: 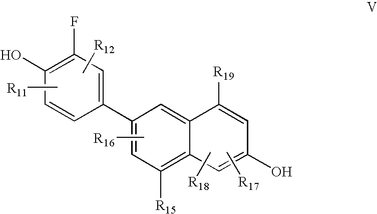
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈ or R₉ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen, or a pharmaceutically acceptable salt thereof;
or the Formula VIII: 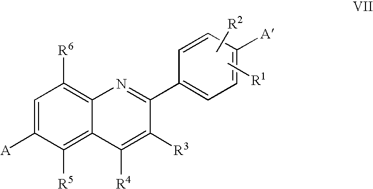
wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C₆ alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, or heteroaryl;
R⁵ and R⁶ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H;
or a N-oxide thereof;
or the Formula X: 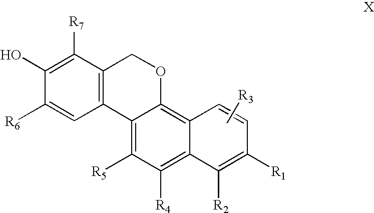
wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁, or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl; wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt or prodrug thereof.

42. The method of claim 6, wherein the ERβ selective ligand is a compound having the formula: 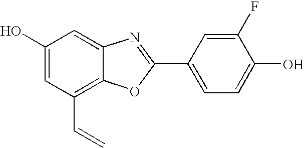
or a pharmaceutically acceptable salt thereof;
or the formula: 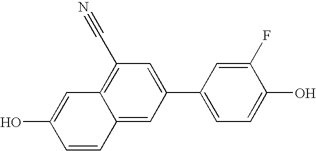
a pharmaceutically acceptable salt thereof;
or the formula: 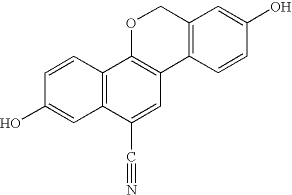
or a pharmaceutically acceptable salt thereof.

43. The method of claim 14, wherein the ERβ selective ligand has the Formula I: 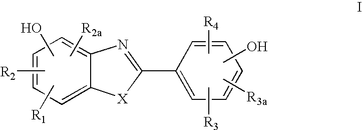
wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
or the Formula II: 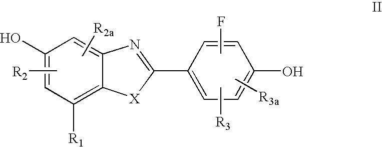
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
or
the Formula III: 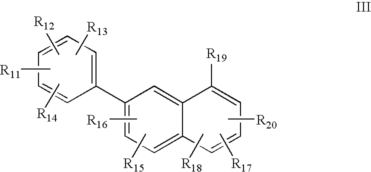
wherein:
R₁₁, R₁₂, R₁₃, and R₁₄ are each, independently, selected from hydrogen, hydroxyl, alkyl of 1-6 carbon atoms, alkoxy of 1-6 carbon atoms, or halogen;
R₁₅, R₁₆, R₁₇, R₁₈, R₁₉, and R₂₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, R₁₉, or R₂₀ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₁, R₁₂, R₁₃, R₁₄, R₁₇, R₁₈, R₁₉ or R₂₀ is hydroxyl, or a pharmaceutically acceptable salt thereof;
or
the Formula IV: 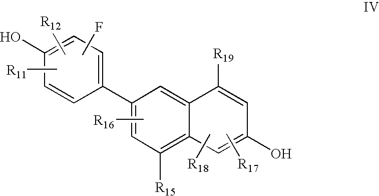
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen, or a pharmaceutically acceptable salt thereof;
or
the Formula V: 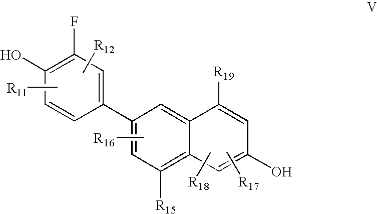
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈ or R₉ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen, or a pharmaceutically acceptable salt thereof;
or the Formula VII: 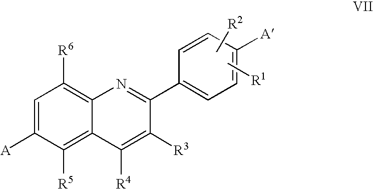
wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C₆ alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, or heteroaryl;
R⁵ and R⁶ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H;
or a N-oxide thereof;
or the Formula X: 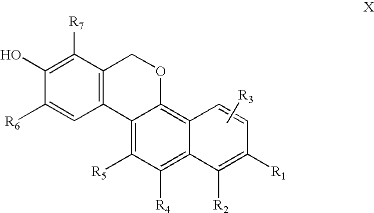
wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁ or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl; wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt or prodrug thereof.

44. The method of claim 14, wherein the ERβ selective ligand is a compound having the formula: 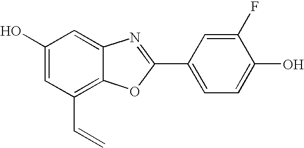
or a pharmaceutically acceptable salt thereof;
or the formula: 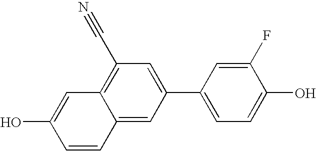
or a pharmaceutically acceptable salt thereof;
or the formula: 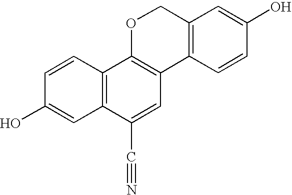
or a pharmaceutically acceptable salt thereof.

45. The method of claim 21, wherein the ERβ selective ligand has the Formula I: 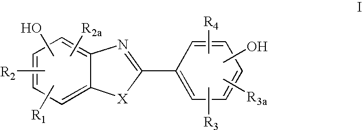
wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
or the Formula II: 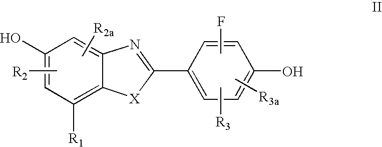
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
or
the Formula III: 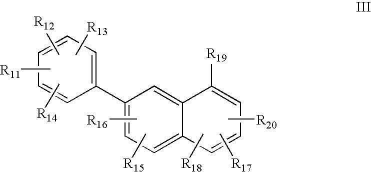
wherein:
R₁₁, R₁₂, R₁₃, and R₁₄ are each, independently, selected from hydrogen, hydroxyl, alkyl of 1-6 carbon atoms, alkoxy of 1-6 carbon atoms, or halogen;
R₁₅, R₁₆, R₁₇, R₁₈, R₁₉, and R₂₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, R₁₉, or R₂₀ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₁, R₁₂, R₁₃, R₁₄, R₁₇, R₁₈, R₁₉ or R₂₀ is hydroxyl, or a pharmaceutically acceptable salt thereof;
or
the Formula IV: 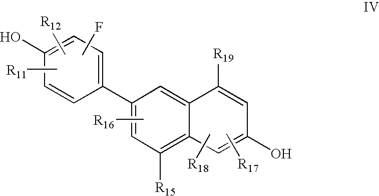
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen, or a pharmaceutically acceptable salt thereof;
or
the Formula V: 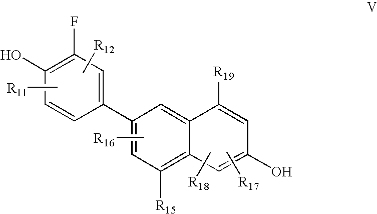
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈ or R₉ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen, or a pharmaceutically acceptable salt thereof;
or the Formula VII: 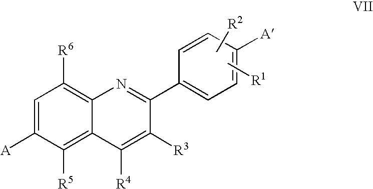
wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C₆ alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, or heteroaryl;
R⁵ and R⁶ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H;
or a N-oxide thereof;
or the Formula X: 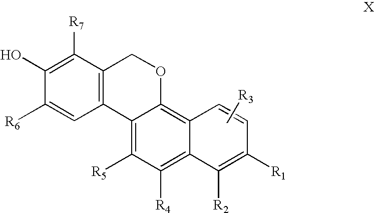
wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁, or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl; wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt or prodrug thereof.

46. The method of claim 21, wherein the ERβ selective ligand is a compound having the formula: 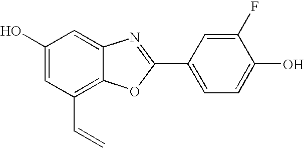
or a pharmaceutically acceptable salt thereof;
or the formula: 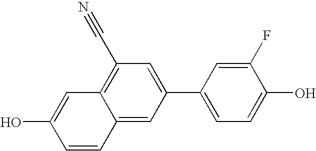
or a pharmaceutically acceptable salt thereof;
or the formula: 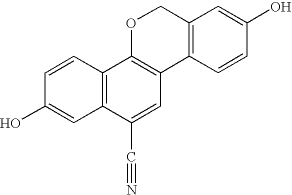
or a pharmaceutically acceptable salt thereof.

47. The method of claim 24, wherein the ERβ selective ligand has the Formula I: 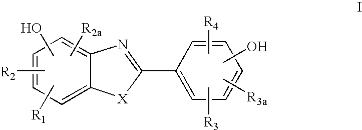
wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
or the Formula II: 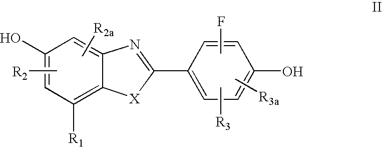
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
or
the Formula III: 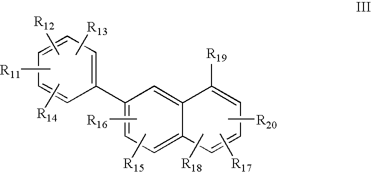
wherein:
R₁₁, R₁₂, R₁₃, and R₁₄ are each, independently, selected from hydrogen, hydroxyl, alkyl of 1-6 carbon atoms, alkoxy of 1-6 carbon atoms, or halogen;
R₁₅, R₁₆, R₁₇, R₁₈, R₁₉, and R₂₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, R₁₉, or R₂₀ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₁, R₁₂, R₁₃, R₁₄, R₁₇, R₁₈, R₁₉ or R₂₀ is hydroxyl, or a pharmaceutically acceptable salt thereof;
or
the Formula IV: 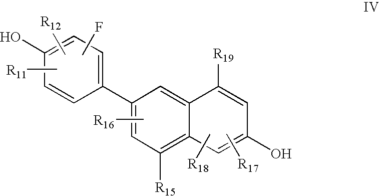
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen, or a pharmaceutically acceptable salt thereof;
or
the Formula V: 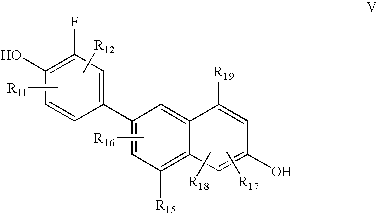
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈ or R₉ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen, or a pharmaceutically acceptable salt thereof;
or the Formula VII: 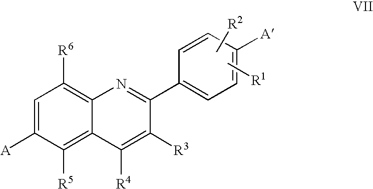
wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C₆ alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, or heteroaryl;
R⁵ and R⁶ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H;
or a N-oxide thereof;
or the Formula X: 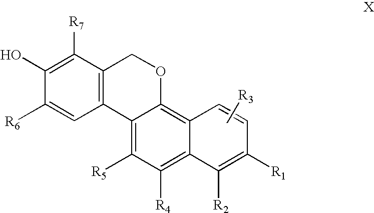
wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁, or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl; wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt or prodrug thereof.

48. The method of claim 24, wherein the ERβ selective ligand is a compound having the formula: 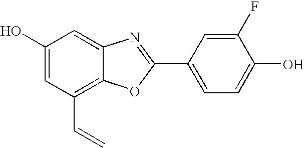
or a pharmaceutically acceptable salt thereof;
or the formula: 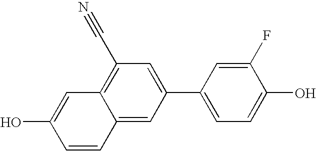
a pharmaceutically acceptable salt thereof;
or the formula: 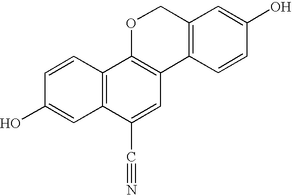
or a pharmaceutically acceptable salt thereof.
